(19) United States
(12) Patent Application Publication
(10) Pub. No.: US 2014/0187945 A1
(43) Pub. Date: Jul. 3, 2014

(54) Ultrasound Probe

(71) Applicants: Cochran Sandy, Dundee (GB); Roderick Habeshaw, Dundee (GB); Graeme McLeod, Dundee (GB); George Corner, Dundee (GB); Jonathan Marsh, Brechin (GB)

(72) Inventors: Cochran Sandy, Dundee (GB); Roderick Habeshaw, Dundee (GB); Graeme McLeod, Dundee (GB); George Corner, Dundee (GB); Jonathan Marsh, Brechin (GB)

(73) Assignee: UNIVERSITY OF DUNDEE, Dundee (GB)

(21) Appl. No.: 14/240,419

(22) PCT Filed: Aug. 24, 2012
(86) PCT No.: PCT/GB2012/052096

§ 371 Date: Feb. 24, 2014

(30) Foreign Application Priority Data
Nov. 3, 2011 (GB) 1119005.5

(51) Int. Cl. A61B 8/00; A61M 19/00; G01S 7/52

(52) U.S. Cl. CPC A61B 8/4477; G01S 7/52017; A61M 19/00
USPC 600/439; 367/7

(57) ABSTRACT

An ultrasound probe (10) comprises a pair of one dimensional transducer modules (1, 2) arranged in-line but set apart from each other in a shallow “V” shape. This allows a wide gap for needle manipulation while maintaining the ability to compute a good acoustic image of the target area (4) on an ultrasound scan engine. The ultrasound image is a product of data from both the arrays. The probe (10) is shaped to allow ergonomic manipulation possibilities for the anaesthetist. The probe can be held in a ‘pencil-type’ grip anywhere on the main body, or by pushing directly on the back of arrays. The shape of the probe, means it sits in position better on a patient than a conventional probe.

### FIELD OF THE INVENTION

The present invention relates to an ultrasound imaging probe for use in ultrasound guided interventional procedures including regional anaesthesia.

### BACKGROUND OF THE INVENTION

Current techniques in ultrasound guided regional anaesthesia (UGRA) require an anaesthetist to position a needle tip adjacent to a nerve bundle and inject anaesthetic agent under the guidance of medical ultrasound imaging. In order to maintain a clearer image of the needle tip position the needle may be inserted ‘in-plane’ with the array.

In order to insert a needle in-plane, as directly to target as possible and avoiding passing the needle along and under the length of the array; previous inventions have involved taking a linear array and removing elements from the central section to create a gap for needle insertion.

This technique may create an unwanted gap in the ultrasound image over the area of greatest interest.

It also does not aid the visibility of the needle shaft or tip due to the angle of attack between the length of needle and acoustic beam.

Because of a necessity to minimise the gap in the acoustic image, the physical gap for the needle to pass though must also be kept to a minimum, and this reduces possible articulation in the gap with the probe in place.

Curved arrays may allow a more direct entry of the needle but are not generally available in a suitable frequency and for other reasons may not offer optimum imaging or other features.

Previous proposals such as those described in the publication by S. Cochran, G. a Corner, K. J. Kirk, D. I. a Lines, and M. J. Watson, “P5C-5 Design and Validation of an Ultrasound Array Optimised for Epidural Needle Guidance,” 2007 IEEE Ultrasonics Symposium Proceedings, vol. 1, October 2007, pp. 2255-2258; in U.S. Pat. No. 4,408,611 by S. Enjoji entitled “Probe for ultrasonic imaging apparatus”; and in U.S. Pat. No. 4,029,084 by R. Soldner entitled “Ultrasound applicator with guide slot for puncturing cannula” are essentially flat linear arrays with missing elements over the gap where the needle passes through.

Each probe functions as a normal linear (1D) array with a corresponding gap in the image. There is minimal overlap in the acoustic image from the two blocks of elements as all elements exist on the same linear plane. The direction/plane of propagation of each block of elements is identical.

Other inventions (U.S. Pat. No. 4,475,553 by Yamaguchi et al.) have addressed the issues associated with the gap with extra laterally displaced imaging components; however this adds complication to the probe design and image reconstruction and still allows only minimal articulation in the gap.

U.S. Pat. No. 6,423,002 by J. A. Hossack, entitled “Intra-operative diagnostic ultrasound multiple-array transducer probe and optional surgical tool” and U.S. Pat. No. 7,214,191 by B. Stringer and G. Simmons entitled “Multiplanar ultrasonic vascular imaging device, system incorporating same, method of use and protective sheath,” are probes where at least two individual blocks of elements are arranged onto the same 2D plane and lie perpendicular to each, i.e. in a T-shape. Each block of elements produces an individual image which can be viewed adjacent to each other or in pseudo (wrap-around) 3D, on-screen.

Again there is no significant overlap in imaging area. The direction of propagation of each block of elements is the same, while the two acoustic imaging fields lie on perpendicular planes.

A related U.S. Patent 2005020919A by B. J. Stringer, G. A. Simmons, D. A. Christensen, S. Messerly, C. P. Ford, and R. W. Evensen entitled “Multiplanar ultrasonic vascular sensor assembly, system and methods employing same, apparatus for movably affixing a sensor assembly to a body and associated methods,” is the same as above, except that it has the addition of a single/double element angled towards the imaging field for doppler analysis.

The angled element is not an array. Although it is defined as comprising of at least one element, it has only one element in cross section along the plane of the adjacent array.

A publication by M. Delaide and G. Maes entitled “Design and Application of Low-Frequency Twin Side-by-Side Phased Array Transducers for Improved UT Capability on Cast Stainless Steel Components,”Proc. 2nd Int. Conf. on NDE in Relation to Structural Integrity for Nuclear and Pressurized Components, New Orleans May 2000, describes the use of 2×2D arrays consisting of 4×2 elements positioned directly next to each other and angled inwards.

The main differences are:

1/ The arrays described are 2 dimensional, i.e. each is arranged in a grid of elements, in this case 2×4 elements with only 2 elements in lateral section.

2/ There is no gap between the edges of the adjacent arrays

3/ The probe is connected to a shaped waveguide for mounting to steel.

4/ The intended aim is to improve beam focusing rather than provide an overlapping visual image area.

It is an object of at least one aspect of the present invention to obviate or mitigate at least one or more of the aforementioned problems.

### SUMMARY OF THE INVENTION

According to a first aspect of the present invention there is provided an ultrasound probe comprising a pair of transducer modules arranged in-line and spaced apart from each other to form a shallow “V” shape in a defined range of angles which correspond to ergonomic conformity to a patient's body surface at procedure specific locations such as to allow a full ultrasound image of a target depth through an ultrasound scan engine.

Preferably the ultrasound probe is shaped to conform to the body surface of the majority of patients over the areas of the neck, axilla, forearm, lower leg, chest wall, knee and other joints.

Preferably also the angle between each front plane of the twin arrays is angled between 110° and 140°, conforming to the respective part of the body being imaged.

The ultrasound probe preferably provides a gap for needle manipulation through the center of the two arrays of between 2 mm and 15 mm.

Preferably the ultrasound image is created through a recombination of waveform data obtained from the separate arrays by treating the twin angled arrays as one virtual array, using respective delays and angular realignment of the individual transmit and receive channels to create one continuous visual image.

More preferably the ultrasound image creation includes the application of beam steering and RF signal compounding to the arrays to produce an improved visual image.

Alternatively the recombination of waveform data is obtained from the separate arrays by capturing standard B-mode images from each array individually, rotating and positioning said images, using weighted pixel selection and overlapping visual gain mixing to create one cohesive image.

The arrays may be straight linear arrays or curved.

The ultrasound probe may be shaped to allow ergonomic manipulation possibilities for an operator.

A third array may be provided arranged perpendicular to the plane of the first pair, at a similar angle thereto and also in-plane with a needle entry area, which adds a concurrent 2D image to the combined image.

According to a second aspect of the present invention there is provided a method of carrying out ultrasound guided regional anaesthesia using an ultrasound probe in accordance with a first aspect of the invention.

### BRIEF DESCRIPTION OF THE DRAWINGS

Embodiments of the present invention will now be described, by way of example only, with reference to the accompanying drawings in which:

FIG. 1 is a representation of the layout and operating range of an ultrasound probe according to an embodiment of the present invention having flat arrays;

FIG. 2 is a front view of an ultrasound probe similar to that of FIG. 1 arranged to provide an expanded field of view;

FIG. 3 is a corresponding view to FIG. 1 illustrating the layout and operating range of an ultrasound probe having curved arrays;

FIG. 4 is a perspective view from below of an ultrasound probe in accordance with the present invention;

FIG. 5 is a front perspective view from above of the ultrasound probe of FIG. 4;

FIG. 6 is a perspective view from below of an alternative embodiment of an ultrasound probe in accordance with the present invention;

FIG. 7 is a representation of the visual display resulting from use of an ultrasound probe as exemplified in FIGS. 1-5; and

FIG. 8 is a representation of the visual display resulting from an ultrasound probe as exemplified in FIG. 6.

### BRIEF DESCRIPTION

Referring to the drawings an ultrasound probe 10 of the present invention comprises a pair of one dimensional transducer modules 1, 2, arranged in-line but set apart from each other in a shallow “V” shape.

This allows a wide gap for needle manipulation while maintaining the ability to compute a good acoustic image of the target area 4 on the ultrasound scan engine.

The ultrasound image is a product of data from both the arrays.

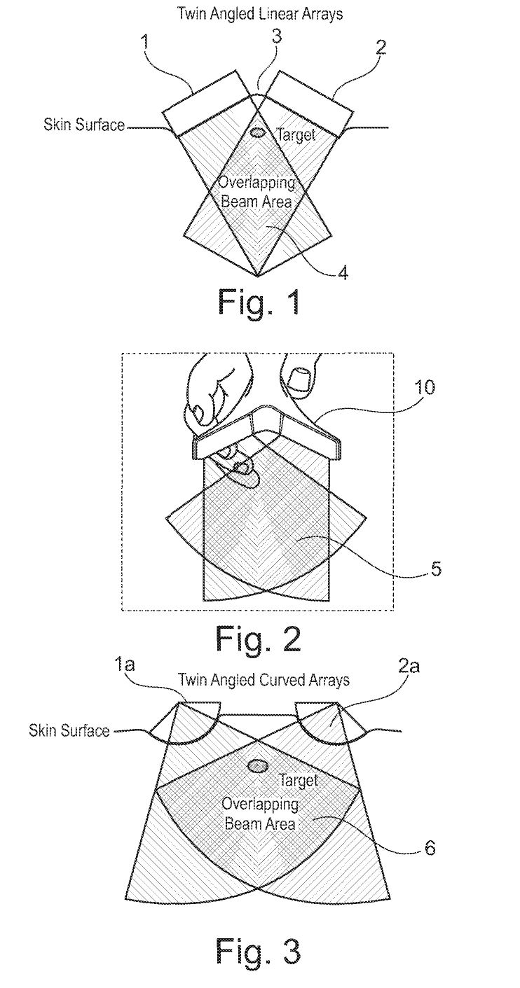

The concept can be applied to linear arrays as shown in FIG. 1, or for a wider field of view 5, using expanded field of view algorithms as in FIG. 2 or a pair of curved linear arrays 1a, 2a as in FIG. 3.

The probe 10 is shaped to allow ergonomic manipulation possibilities for the anaesthetist. The probe can be held in a ‘pencil-type’ grip anywhere on the main body, or by pushing directly on the back of arrays.

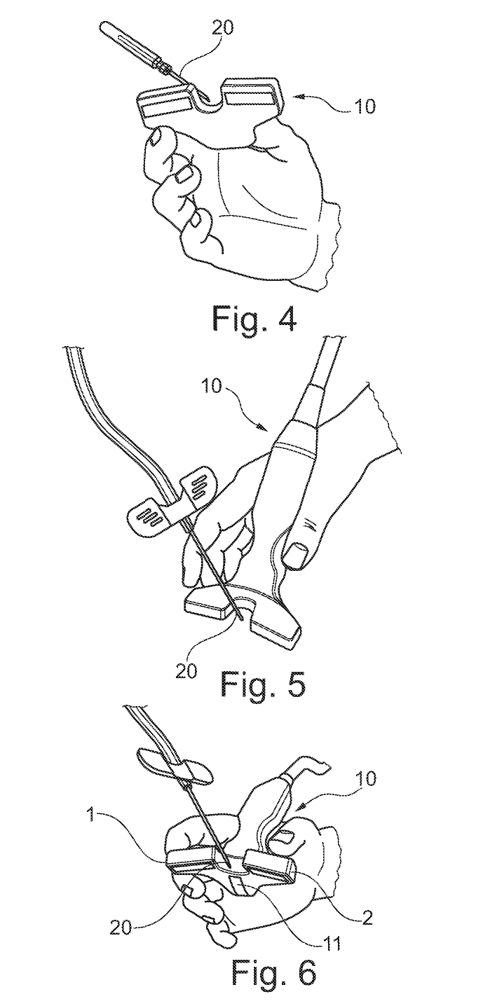

The shape of the probe, which can best be seen in FIGS. 2, 4 & 5 means it sits in position better on a patient than a conventional probe.

A further option is the addition of a third array 11 arranged perpendicular to the plane of the twin pair and in-plane with the needle entry 20 as shown in FIG. 6; this gives a secondary in-plane view of the target area and needle point . The third array is set at a similar angle to the others.

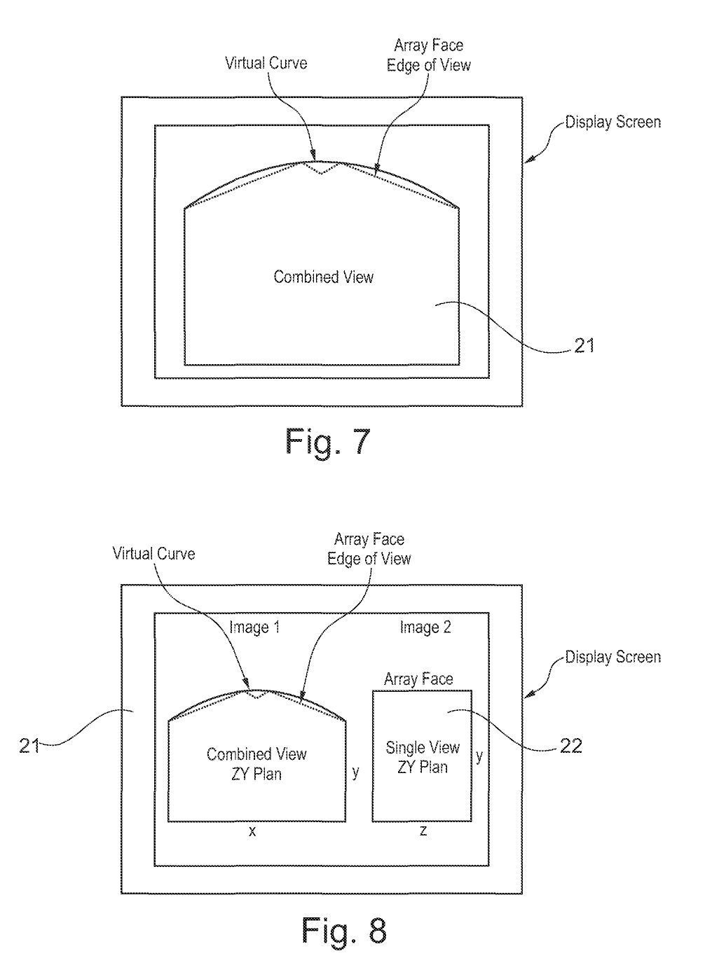

In the case of the twin array, a visual display will be constructed using the data from both array modules, combined with scan engine processing to form one cohesive image 21 covering the extent of the acoustic beam width of the probe as can be seen in FIG. 7.

With the triple array probe, a third array image 22 will be displayed side by side with the combined image 21 from the twin arrays as can be seen in FIG. 8.

The image reconstruction for the combined view can be performed either through visual mixing of the independent images from each array or by forming one visual image through the interpretation of transmit and receive signals to represent one virtual curved or linear array.

The ultrasound probe of the current invention has a number of advantages.

In particular a pair of arrays are set apart and angled in a shallow ‘V’ shape with overlapping beam area providing an ultrasound image.

The conformal shape of the device provides a more stable ‘platform’ for an anaesthetist.

The probe does not provide a ‘needle guide’ as such and the anaesthetist is free to work within an extended gap.

Optionally the inclusion of a perpendicular third array, also angled enhances the probe.

The probe may incorporate ‘sparse’ array beam forming to utilise the full width of all available array elements despite the limitations of the maximum number of independent channels that current circuitry can handle.

Whilst specific embodiments of the present invention have been described above, it will be appreciated that departures from the described embodiments may still fall within the scope of the present invention.

Throughout the description and claims of this specification, the words “comprise” and “contain” and variations of them mean “including but not limited to”, and they are not intended to (and do not) exclude other components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers and characteristics described in conjunction with a particular aspect, embodiment or example of the invention are to be understood to be applicable to any other aspect, embodiment or example described herein unless incompatible therewith.

All of the features disclosed in this specification (including any accompanying claims, abstract and drawings), and/or all of the steps of any method or process so disclosed, may be combined in any combination, except combinations where at least some of such features and/or steps are mutually exclusive.

The invention is not restricted to the details of any foregoing embodiments.

The invention extends to any novel one, or any novel combination, of the features disclosed in this specification (including any accompanying claims, abstract and drawings), or to any novel one, or any novel combination, of the steps of any method or process so disclosed.

1. An ultrasound probe comprising a pair of transducer modules arranged in-line and spaced apart from each other to form a shallow “V” shape, said shallow “V” shape configured to provide a gap for needle manipulation while allowing the creation of a full ultrasound image of a target area on a standard ultrasound scan engine.

2. An ultrasound probe as claimed in claim 1, wherein each of said pair of transducer modules is an array.

3. An ultrasound probe as claimed in claim 2, wherein the full ultrasound image is a product of data from said arrays.

4. An ultrasound probe as claimed in claim 3, wherein the full ultrasound image of the target area is created through a recombination of a waveform data obtained from the array to create one cohesive image of said full ultrasound image.

5. An ultrasound probe as claimed in claim 4, wherein the recombination of the waveform data is obtained from the array by treating the array as a virtual array, using respectively a delays and an angular realignment of a transmit and a receive channel of the array to create said one cohesive image.

6. An ultrasound probe as claimed in claim 4, wherein the recombination of waveform data is obtained from two of the array by capturing a standard B-mode image from each of said two of the array individually, a means of manipulating and joining said standard B-mode images by an image processing algorithm to create the one cohesive image.

7. An ultrasound probe as claimed claim 6 wherein the arrays is configured to be flat or curved.

8. An ultrasound probe as claimed in claim 7, wherein the ultrasound probe is shaped to enable an ergonomic manipulation of the ultrasound probe.

9. An ultrasound probe as claimed in claim 8, wherein the ultrasound probe is shaped to conform to a body shape of a patient.

10. An ultrasound probe as claimed in claim 9, wherein a third array may be provided arranged perpendicular to the plane of the other two-array, at a similar angle thereto and also in-plane with a needle entry area.

11. A method of carrying out ultrasound guided regional anaesthesia using an ultrasound probe in accordance with claim 10.
